(19) United States
(12) Patent Application Publication
(10) Pub. No.: US 2003/0130169 A1
(43) Pub. Date: Jul. 10, 2003

(54) Methods of treating drug-resistant bacterial infections

(75) Inventors: Paul J. Hergenrother, Champaign, IL (US); Dinty J. Musk JR., Champaign, IL (US); Johna C.B. DeNap, Rantoul, IL (US)

Correspondence Address: SONNENSCHEIN NATH & ROSENTHAL, P.O. BOX 061080, WACKER DRIVE STATION, CHICAGO, IL, 60606-1080, US

(73) Assignee: The Board of Trustees of the University of Illinois

(21) Appl. No.: 10/261,851

(22) Filed: Oct. 1, 2002

Related U.S. Application Data
(60) Provisional application No. 60/326,315, filed on Oct. 1, 2001.

(51) Int. Cl. A61K 38/17; A61K 38/14; A61K 31/7048; A61K 31/704; A61K 31/496; A61K 31/4709; C12Q 1/68

(52) U.S. Cl. 514/2; 514/8; 514/29; 514/152; 514/35; 514/253.08; 514/312; 514/255.06; 514/155; 514/376; 435/6

(57) ABSTRACT

Methods for treatment of antibiotic-resistant and multi-drug resistant bacterial infections are provided. The methods comprise administration of compositions which mimic plasmid incompatibility in bacteria, resulting in their sensitization to previously resistant drugs. Also provided herein are methods for screening compositions for the ability to mimic plasmid incompatibility by inhibiting Rep protein or by destabilizing RNA/RNA stem loop “kissing” structures. The invention also encompasses compositions identified by the screening methods disclosed herein.

This application claims priority to U.S. provisional patent application Serial No. 60/326,315, filed Oct. 1, 2001, the contents of which are hereby incorporated by reference.

The invention was supported by grant N00014-02-1-0390 from the Office of Naval Research. The government has certain rights in the invention.

### FIELD OF THE INVENTION

The present invention relates to methods of treating antibiotic-resistant bacterial infections, including multi-drug resistant bacterial infections. The methods relate to administration of antiplasmid compositions which have the ability to mimic plasmid incompatibility, thereby leading to the loss of the plasmid(s) from bacterial cells and sensitization of the bacteria to the drugs to which they were previously resistant. The invention also encompasses methods for screening compounds for the ability to mimic plasmid incompatibility.

### BACKGROUND OF THE INVENTION

Despite the invention of antibiotics, bacterial infections still claim many lives worldwide. Furthermore, during the last several decades bacterial resistance has emerged as a new trend, contributing to morbidity and mortality caused by bacterial infections. A troubling percentage of all infections encountered in clinical settings are resistant to some form of antibiotic therapy. Due to the excessive and not always appropriate use of antibiotics in humans and animal feed, bacterial resistance currently constitutes a major public health crisis. The World Health Organization (WHO) reported that drug resistant strains of microbes had a negative impact on their fight against tuberculosis, cholera, diarrhea and pneumonia, which together killed more than ten million people worldwide in 1995 (Molnar, J., 1997).

Multi-drug resistant strains of bacteria such as methicillin-resistant Staphylococcal aureus (MRSA) and vancomycin-resistant enterococci (VRE) were first encountered in hospital settings, but many of them can now be found infecting healthy individuals in larger communities. The spread of VRE is particularly concerning when it is taken into account that vancomycin is generally regarded as the last line of defense in the antibiotic arsenal. Furthermore, the extensive use of beta-lactam antibiotics such as penicillin and ampicillin has also resulted in significant numbers of resistant strains among both Gram-positive and Gram-negative bacteria.

Currently, the choices for treatment of antibiotic-resistant and multi-drug resistant bacteria are limited in scope even though the molecular mechanisms of resistance are fairly well understood. In many cases, antibiotic-resistant and multi-drug resistant bacteria such as MRSA and VRE encode the antibiotic resistance genes on plasmids. These plasmids can be laterally transferred between bacteria and hence account for the rapid dissemination of antibiotic resistance genes into diverse bacterial populations. These plasmid genes, which often encode enzymes that metabolize antibiotics are responsible for reducing the intracellular accumulation of drugs and ineffectiveness of drug treatments.

Plasmids are thus very effective at maintaining and spreading drug resistance among bacteria. They are autonomous, self-replicating extra-chromosomal DNA molecules that are generally not essential for survival of bacteria, but which encode for a variety of factors beneficial for bacterial survival in adverse conditions. The plasmids are present in defined copy numbers in bacterial cells, and their replication largely depends on host-encoded factors. Despite the vast numbers of different plasmids, their regulation of replication can generally be categorized into two groups. The first method of replication control is based on the regulation of Rep protein, which is encoded by a plasmid rep gene. The Rep protein plays a critical role in plasmid replication due to its phosphodiesterase activity at the origin of replication (ori), which allows replication to initiate. This catalytic activity of Rep is inhibited by short (20 bp) direct repeats of DNA located near the origin of replication or in proximity to the Rep gene. The direct repeats bind to Rep, inhibiting its phosphodiesterase activity and resulting in attenuated plasmid replication.

The second method of replication control is achieved through the use of short RNA oligonucleotides which function as primers in plasmid replication. More specifically, bacteria harboring the plasmid synthesize small RNA molecules that are anti-sense to the RNA primers and can thus bind to them. This binding between an RNA primer and its antisense RNA molecule occurs through a stem-loop “kissing” complex, which leads to inhibition of replication.

Another important factor in plasmid replication is the compatibility of two or more plasmids in a bacterial cell. It is now known that plasmids can be classified into various incompatibility groups, wherein the plasmids that can not co-exist in the same cell are placed in the same incompatibility group. The molecular basis for plasmid incompatibility is relatively well understood. When two or more plasmids from the same incompatibility group occur in the same cell, their similar mode of replication will force them to compete for various proteins and oligonucleotides involved in the replication. A plasmid that has a higher affinity for these various factors will be able to sequester them and use them to replicate, whereas the competing plasmid will be prevented from doing so, eventually leading to the loss of unreplicated plasmid from the cells.

In recent years, there have been attempts to discover new drugs for treatment of bacterial infections, especially for growing numbers of bacterial infections caused by multiple-drug resistant strains. The vast majority of antibiotics that have been introduced in the market in the last two decades are derivatives of previously available antibiotics. Until the emergence of oxazolidinones in late 1999, a new structural class of antibiotics had not been introduced since fluoroquinolones in the mid-1970's. Although derivatizing known antibiotics has in many cases proven to be successful in eliminating unwanted side effects or enhancing pharmacokinetic properties, bacteria that are resistant to one drug are often resistant to its derivatives. The lack of new drugs targeting drug-resistant bacteria has also led to performance of bactericidal tests with drugs other than antibiotics and their derivatives.

Phenothiazines, which had been used to treat central nervous system disorders were found to possess antibacterial effect on a wide variety of Gram-positive and Gram-negative bacteria. Their antibacterial effect resulted from their ability to increase permeability of the bacterial cell wall, suggesting a possible use in patients. In cases of beta-lactam antibiotic resistance, the novelty of treatment lay in the introduction of beta-lactamase inhibitors. These inhibitors prevent the activity of the enzyme beta-lactamase encoded by the plasmid, thereby rendering the bacteria harboring the plasmid susceptible to beta-lactam antibiotics. A limitation with this treatment, however, is that such inhibitors are not active against all beta-lactamases.

One development relating to treatment of drug-resistant bacterial infection involves “antiplasmid” compounds. Certain phenothiazines were found to possess antibacterial activity due to their ability to selectively inhibit plasmid replication. Lack of plasmid replication led to the loss (“curing”) of plasmid from the cells, thereby eliminating the resistance of the bacteria. For instance, phenothiazines, stereoisomers of thioxanthenes and enantiomers of mepromazine were found to eliminate various plasmids with different frequencies from cells such as E. coli, Proteus vulgaris, Klebsiella pneumoniae, Yersinia, and Agrobacterium tumefaciens (Molnar J., 1997).

Trovafloxacin (CP-99,219), a member of the fluoroquinolone family, was also found to possess “plasmid curing” effects. This effect was observed with plasmids differing in copy numbers, size and nature of replication.

Accordingly, there is a need to discover novel methods that could successfully combat antibiotic-resistant and multi-drug resistant bacterial infections. In particular, compositions and methods that simulate natural loss of plasmid(s) from cells would be desirable, thereby minimizing the chances of developing new kinds of resistance.

### SUMMARY OF THE INVENTION

Accordingly, among the objects of the invention is the provision of methods for treating drug resistant and multi-drug resistant bacterial infections. Further provided are methods for screening compounds for the ability to interfere with plasmid replication by mimicking plasmid incompatibility.

The methods for treating a drug-resistant bacterial infection include administering to a subject in need of such treatment an effective amount of an antiplasmid composition with the ability to mimic plasmid incompatibility, thereby resulting in “plasmid curing” and sensitization of bacteria to the drug for which resistance is mediated by a plasmid-encoded gene. This is followed by administering to the subject an effective amount of the drug to which bacteria had been sensitized.

The method for treating multi-drug resistant bacterial infections closely resembles the method described above, except that the administration of antiplasmid compounds results in sensitivity of bacteria to multiple drugs. Furthermore, an effective amount of one or more drugs can be administered to the subject following “plasmid curing” in order to eliminate the infection.

For treatments of drug resistant and multi-drug resistant bacterial infections, the subject is preferably a mammal, and even more preferably the subject is a human. In another preferred aspect, the drug is selected from the group of antibiotics consisting of beta-lactams, aminoglycosides, tetracyclins, macrolides, sulfa drugs, lincosamides, glycopeptides, quinolones, amiocyclitols, lincopeptides, polypeptide antibiotics, notroimidazoles, rifampicins, notrofurans, oxazolidinones, trimethoprim, cloramphenicol, isoniazid, methenamine and mupirocin.

In yet another aspect, the antiplasmid composition comprises a composition which mimics plasmid incompatibility, including but not limited to compositions with such activity selected from the group comprising aminoglycosides, such as, e.g., apramycin, tobramycin, paromomycin I, kanamycin B and derivatives thereof. The preferred methods of administration of the compound include subcutaneous injection, intramuscular injection, intravenous administration, inhalation spray, topically, and oral administration.

The infection may be caused by many different bacterial isolates, but preferably the drug resistant bacterial infection is caused by MRSA or VRE.

Methods for screening compounds for the ability to interfere with plasmid replication by mimicking plasmid incompatibility are also provided herein. In one aspect, the method is based on screening compounds for the ability to inactivate the function of Rep protein. Preferably, the Rep protein activity consists of binding of the Rep protein to plasmid DNA sequences. Preferably, the compounds to be screened include aminoglycosides and derivatives thereof. The present invention also provides compositions comprising at least one compound identified by said method of screening.

In another aspect, the method is based on screening compounds for the ability to disrupt a stem-loop interaction between an RNA primer required for plasmid replication and its antisense transcript. Preferably, the compounds include aminoglycosides and derivatives thereof. Further provided are compositions containing at least one compound identified by the method of screening based on disruption of stem-loop interaction.

In another aspect, the invention encompasses a pharmaceutical composition for the treatment of drug-resistant microbes such as drug-resistant strains of bacteria. The composition comprises an effective amount of an antiplasmid composition that mimics plasmid incompatibility, thereby rendering the drug-resistant microbe sensitive to drug or drugs for which resistance is plasmid-mediated and an effective amount of a drug or drugs to which the microbe is sensitized by the antiplasmid composition. Preferably, the pharmaceutical composition comprises a pharmaceutically-acceptable excipient.

In an alternative embodiment, the treatment is provided by means of a kit. The kit comprises a first dosage form comprising an effective amount of an antiplasmid composition that mimics plasmid incompatibility, thereby rendering the drug-resistant microbe sensitive to a drug or drugs for which resistance is plasmid-mediated. The kit also comprises a second dosage form comprising an effective amount of a drug or drugs to which the microbe is sensitized by the antiplasmid composition.

Other objects and features will be in part apparent and in part pointed out hereinafter.

### BRIEF DESCRIPTION OF FIGURES

FIG. 1 depicts the general scheme of the anti-plasmid approach to combating bacterial resistance to antibiotics.

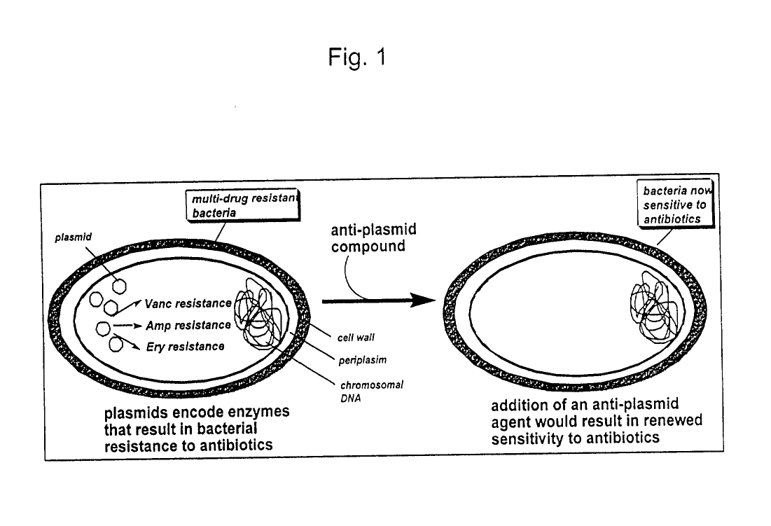

FIG. 2 depicts a scheme of a small molecule action on plasmid replication. 1) represents inhibition of plasmid replication at the level of disrupting RNA/RNA stem-loop “kissing” complex interaction, and 2) represents inhibition of plasmid replication at the level of preventing the binding of Rep protein to direct repeats in the plasmid.

FIG. 3 depicts the general scheme for synthesis of tobramycin (small molecule) core.

FIG. 4 depicts the general scheme for derivatization of an aminoglycoside through deprotection and glycosylation.

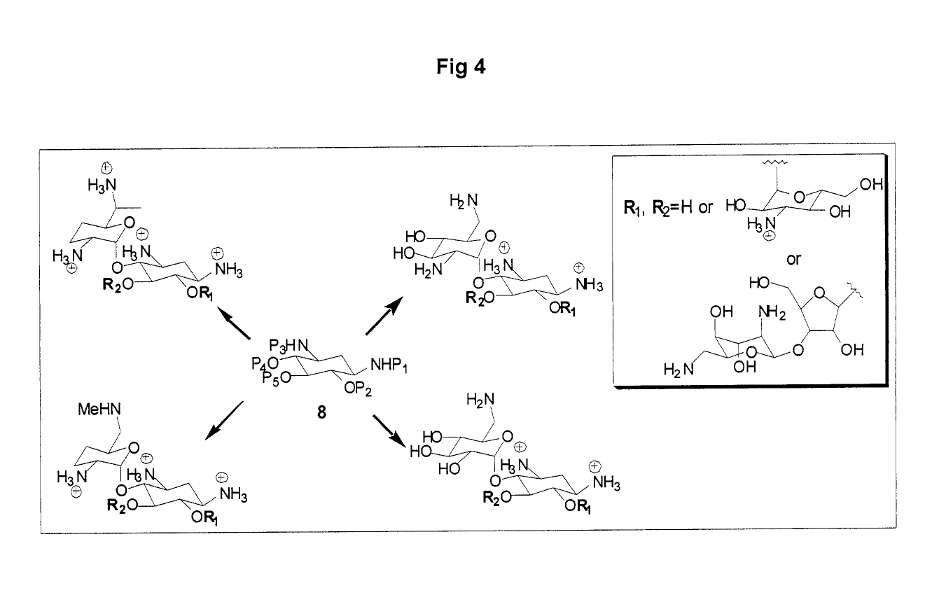

FIG. 5 is a graphic depiction of dose-dependent reductions in the amount of β-galactosidase upon administration of selected small molecules-apramycin (FIG. 5a), paromomycin I (FIG. 5b), and kanamycin B (FIG. 5c) in a RepA-LacZ reporter gene assay which demonstrates loss of plasmid. See Example 1.

FIG. 6 depicts examples of master and replica plates of E. coli containing an IncB plasmid encoding for β-lactamase after growth in the presence of apramycin after 140 generations. Apramycin causes plasmid elimination from E. coli. E. Coli harboring an IncB plasmid encoding for β-lactamase was grown in the presence of apramycin (25 μg/mL) after a defined number of generations (Master Plates). Replica plates were then made onto LB/Apr 12/Amp. Depicted examples are after 140 generations in the presence of apramycin. See Example 2.

FIG. 7 is a graphic depiction of plasmid loss as a function of bacterial generation. The plasmid is virtually eliminated in the presence of apramycin. See Example 2.

FIG. 8 is a graphic depiction of the binding of apramycin to SLI and SLIII. Fluorescein-labeled SLI was titrated with various apramycin concentrations and gross fluorescein measured (solid squares). An identical experiment was then performed with fluorescein-labeled SLIII (open triangles). See Example 3.

### ABBREVIATIONS AND DEFINITIONS

To facilitate understanding of the invention, a number of terms are defined below:

The term “antiplasmid” as used herein refers to the ability to inhibit plasmid replication or function.

The phrase “plasmid curing” as used herein refers to the ability to eliminate a plasmid from a bacterial cell or inactivate said plasmid.

The terms “compounds” and “small molecules” are used interchangeably herein.

### DETAILED DESCRIPTION OF THE INVENTION

In accordance with the present invention, applicants have devised novel methods for treating drug-resistant and multi-drug resistant bacterial infections. These methods include administration of antiplasmid compounds, which have the ability to mimic plasmid incompatibility, thereby leading to loss or inactivation of plasmid(s) from bacterial cells. Due to the fact that most of the antibiotic resistance genes are located within plasmid sequences, the loss of plasmid(s) from bacteria results in sensitization of said bacteria to antibiotics for which resistance is plasmid-mediated.

Plasmid incompatibility is a natural process that occurs in bacteria, whereby a plasmid is eliminated from the cell due to its inability to replicate properly. By mimicking this natural process to treat infections, the mutation rate of bacteria and the development of drug resistance may be inhibited. Furthermore, the methods of this invention allow the use of traditional antibiotics to treat formerly drug-resistant strains without the need to develop new antibiotics.

The invention further encompasses methods of screening compounds for the ability to interfere with plasmid replication by mimicking plasmid incompatibility. The compounds are screened and selected based on their ability, e.g., to inhibit Rep protein activity or disrupt stem-loop interaction between an RNA primer needed for plasmid replication and its antisense transcript.

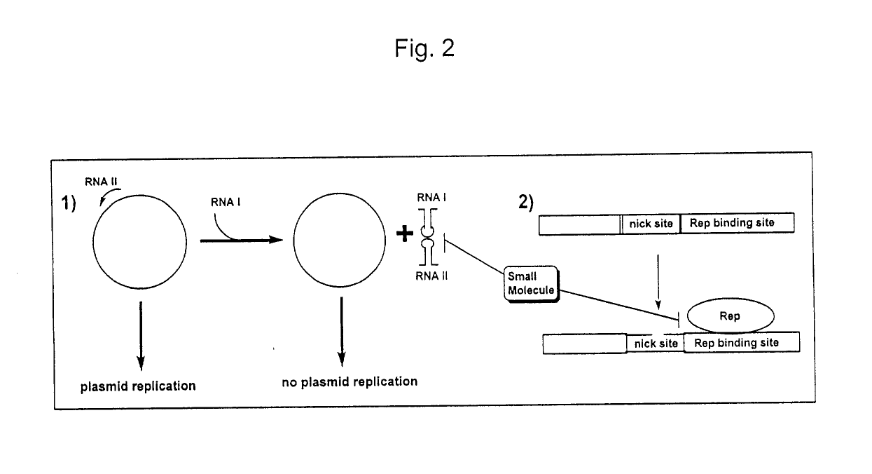

The stem-loop “kissing” interaction, depicted in FIG. 2 plays an important role in plasmid replication. While not being bound to any particular theory, disruption of this RNA/RNA interaction with a small molecule that binds to the “kissing” complex leads to runaway plasmid transcription, and hence plasmid instability and elimination from the cell.

Thus, one aspect of screening compounds for the ability to mimic plasmid incompatibility involves screening of the compounds for the ability to disrupt these stem-loop complexes between an RNA primer required for plasmid replication and its antisense transcript. In one embodiment, small molecules capable of binding RNA with high affinity are screened. In particular, aminoglycosides such as apramycin and tobramycin are screened for their ability to specifically bind to stem-loop structures in RNA. In this embodiment, small molecules may be synthesized and tested for binding to stem-loop “kissing” complexes required for plasmid replication.

Because features of plasmid replication control are common to diverse bacterial strains, the methods of this invention have applicability to a variety of types of bacteria, including both gram positive and gram negative bacteria. In particular, the use of a small piece of RNA, such as RNA I, is commonly employed in plasmid replication control. Moreover, it typically interacts with another stretch of RNA to form loop-loop “kissing” complexes. See FIG. 2. In addition, a consensus sequence—YUNR (Y=pyrimidine, U=uracil, N=any base, R=purine), frequently mediates the critical RNA loop-loop interactions that control plasmid replication. In all cases, this homologous region consists of the first 4 bases on the 5′ side of the loop sequence. RNA loops from 45 different prokaryotic replication control elements were found to contain this YUNR consensus sequence, and this similarity is present in plasmids from an array of incompatibility groups from a variety of bacterial hosts. SLI from the IncB group is among the multitude of RNA loops involved in plasmid replication control that contain this YUNR consensus sequence. The presence of this consensus sequence and the common mechanisms underlying plasmid replication control thus provide a basis for broad application of methods mimicking plasmid incompatibility as described herein.

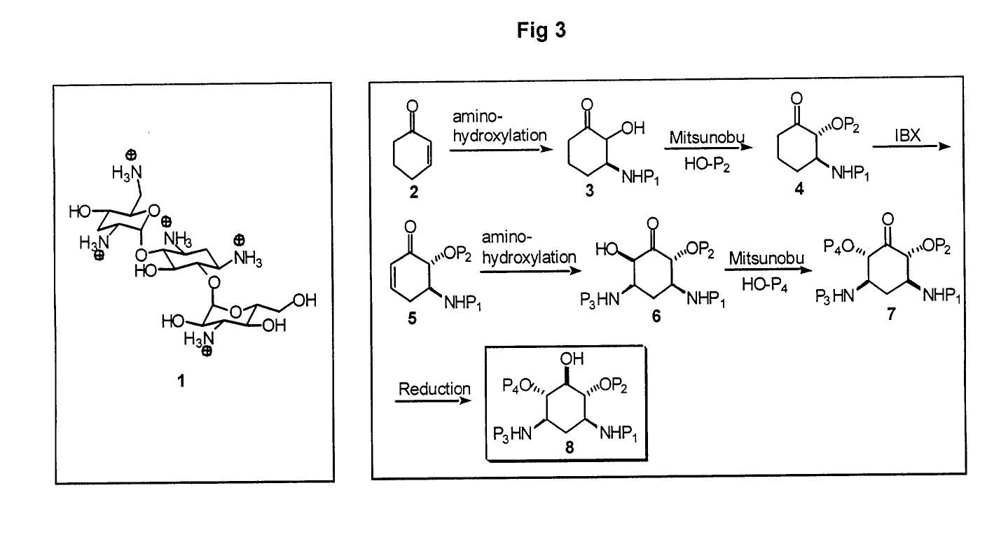

An exemplary method for synthesis of an aminoglycoside antiplasmid composition is described below, and depicted in FIG. 3:

1) the steroselective synthesis of the core structure can begin with the asymmetric aminohydroxylation (AA) as described by Li et al. of the readily available α,β-unsaturated cyclohexanone (marked 2);

2) the regiochemistry of this reaction should be as drawn in 3, as the nitrogen in the AA reaction is known to attach to the carbon atom distal to the electron-withdrawing group (Li et al., 1996);

3) after Mitsunobu inversion and simultaneous protection of the secondary alcohol of 3, the resulting ketone 4 is oxidized to the α,β-unsaturated system 5 using o-iodoxybenzoic acid (IBX) (Nicolaou, 2000), following which this newly created olefin is subjected to another round of the AA reaction followed by inversion/protection to provide 7;

4) axial delivery of a hydride with a small reducing agent such as NaBH₄ stereo-selectively reduces the ketone of 7 to core structure 8.

It should be noted that in case this technique is not satisfactory, longer synthetic routes may be performed to obtain core structure 8. Furthermore, novel compounds can be generated by derivatizing the core structure 8 by differentially deprotecting hydroxyl groups or by appending amino sugars. These sugars are readily available from acid hydrolysis of the natural products (Alper et al., 1998), and many of these natural products are commercially available at low cost. Thus, convenient access to novel compounds may be available through the appropriate glycosylation reactions. Additional novel compounds may be obtained by adjusting these methods or by performing different synthetic routes. The said adjustments and different synthetic pathways can be easily determined by one skilled in the art.

As would be apparent to those skilled in the art, other compounds having binding affinity to RNA are known or may be devised, and their use is considered within the scope of this invention. In accordance with the invention, the compounds are tested for binding to stem-loop “kissing” complexes, e.g., by performing fluorescence assays and determining dissociation constants. See, for example, Wang et al., Chem. Biol., 2:281-290, 1995. The compounds that bind to the “kissing complex” are then examined for antiplasmid effect and subsequent sensitization of bacteria to antibiotics.

The antiplasmid effect can be assayed as follows: Methicillin-resistant Staphylococcus aureus (MRSA) bacteria are transformed with a plasmid containing the genes encoding for the constitutive expression of the β-lactamase and β-galactosidase. On MacConkey agar, colonies expressing an active β-galactosidase are red in color. MRSA containing the plasmid are then plated onto the MacConkey agar plates containing a gradient of concentration of the target compounds. Such plates containing concentration gradients of small molecules have previously been described (Gerhardt et al., 1994), and their preparation is well known in the art. If the compound is an inhibitor of plasmid replication, then as the concentration of the tested compound is increased the number of red colonies decreases. In addition, this assay indicates the potency of the tested compound.

The compounds which exhibited antiplasmid effects can further be tested for the ability to induce “plasmid curing” and sensitization to antibiotics. The exemplary experiments are listed below.

The same MacConkey agar plates are made as described above, except that methicillin is included in the agar at a constant concentration. The concentration of methicillin may, for example, be 100 μg/ml. MRSA transfected with the plasmid (as above) are plated onto these methicillin-containing plates. Accordingly, if the compound has antiplasmid effects and leads to sensitization of bacteria, the number of MRSA colonies should decrease across the plate as concentration of the tested antiplasmid compound is increased. This would be a result of increased reduction of plasmid copy numbers at higher concentrations of the antiplasmid compound, wherein a reduction of plasmid concentration in the bacterial cell leads to increased sensitivity of said cell to the antibiotic (in this case methicillin). It should be noted that the experiments disclosed herein for testing antiplasmid effect of compounds can be modified without effecting their outcome. For instance, the bacteria and antibiotics used in the assays can be changed, and concentrations may be varied. Such modifications are easily determined by one skilled in the art.

The screen may also include a toxicity test, wherein the compounds are tested in mammalian cell survival assays to ensure that they are not toxic to mammalian cells. See for example Stockwell et al., Chem. Biol., 6:71-83, 1999.

In another aspect, the compounds can be tested for the ability to inhibit Rep protein activity. For example, compounds such as aminoglycosides and derivatives thereof are tested for their ability to interfere with Rep protein due to its role during plasmid replication. There are multiple ways to screen small molecules for the ability to bind to Rep and inhibit its phosphodiesterase function. For example, small molecule microarrays can be formed and then tested for binding to Rep. Briefly, small molecules are covalently attached to glass slides, and allowed to interact with fluorescently-labeled Rep. After washing the slides, detection of a fluorescent signal on the slide indicates the ability of specific molecules to bind to Rep. One skilled in the art can readily design and perform said assays.

Once the successful binders are identified, they are tested for antiplasmid and “plasmid curing” activities and finally, if they possess such activity they can be further tested for toxicity to mammalian cells. These assays are essentially identical to the assays described above.

The compound(s) that are obtained from either screen, i.e. the compounds that have antiplasmid activity, sensitize drug resistant bacteria to traditional antibiotics and are not toxic to mammalian cells at appropriate dosages may be used to treat subjects suffering from drug-resistant bacterial infections, including multi-drug resistant ones. Thus, compositions are provided consisting of at least one compound identified by such screening method.

Briefly, the method of treating drug resistant bacterial infections, including multi-drug resistant infections consists of 1) administering to a subject suffering from a drug-resistant bacterial infection an effective amount of an antiplasmid composition that mimics plasmid incompatibility, thereby rendering the drug-resistant bacteria sensitive to the drug(s) for which the resistance is plasmid-mediated, and 2) administering to the subject an effective amount of the drug(s) to which bacteria has been sensitized.

In one embodiment of the method of the invention, a sub-lethal dose of an antiplasmid composition is administered to render the drug-resistant bacteria sensitive to the drug or drugs for which the resistance is plasmid-mediated. This allows the formerly drug-resistant bacteria to be treated by administration of one or more drugs to which the bacteria has been sensitized. For example, a bacteria with a plasmid-mediated resistance to ampicillin may be administered sub-lethal doses of apramycin to eliminate the ampicillin resistance, and then administered ampicillin to treat the sensitized bacteria. See Example 2 below.

The drug-resistant bacterial infection may be caused by any bacterial strain, resistant to at least one antibiotic, including ones caused by MRSA and VRE.

In another aspect, the drug is selected from the group of antibiotics including beta-lactams, aminoglycosides, tetracyclins, macrolides, sulfa drugs, lincosamides, and glycopeptides. These groups of antibiotics and their constituents are well known in the art. For example, beta-lactams include penicillins such as penicillin G, ampicillin, and amoxiciliin, and cephalosporins such as cephamycin, cefonicid, cefotetan, and cephalothin. Preferably, the subject undergoing this treatment is a mammal, and more preferably the subject is a human.

In another aspect, the compound is selected from the group comprising aminoglycosides, such as tobramycin. In another aspect, the compound is administered by subcutaneous injection, intramuscular injection, intravenous administration or oral administration.

### EXAMPLE 1

### Reporter Gene Assay

Small molecules were tested for their ability to imitate the function of RNA I by binding to the RepA mRNA and disrupting the Stem Loop I-Stem Loop III (SLI-SLIII) loop-loop interaction, thus mimicking plasmid incompatibility. Aminoglycosides and their derivatives were selected for testing because of their ability to tightly bind RNA from a variety of sources, particularly regions of distorted RNA secondary structure, such as RNA loops or bulges.

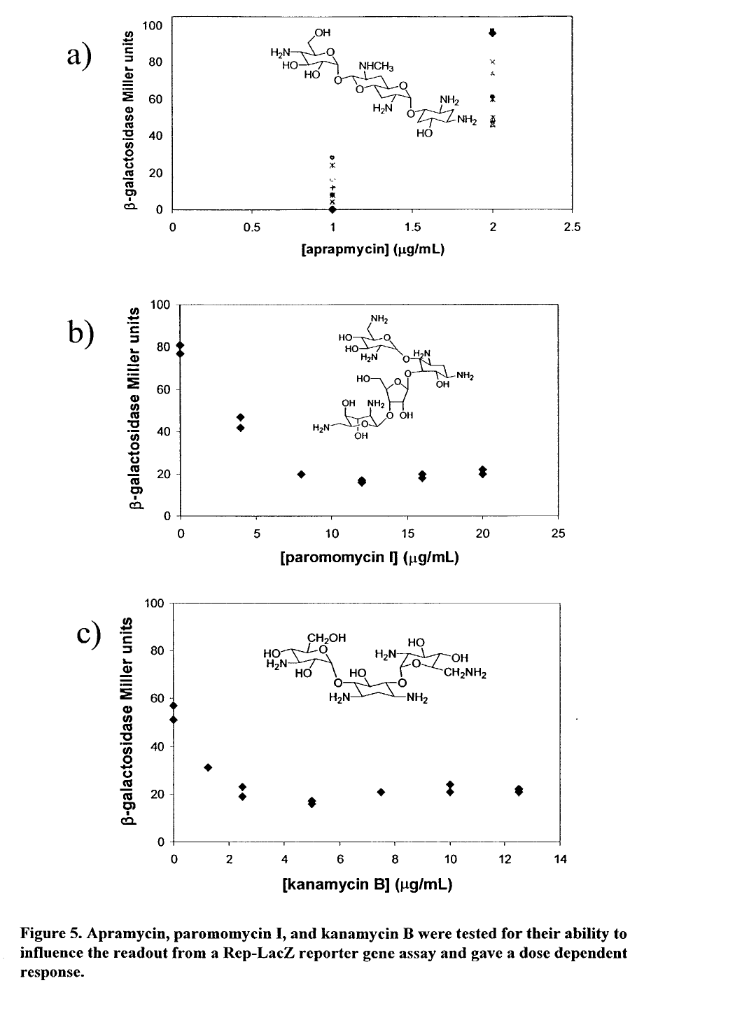

To assess their ability to disrupt the SLI-SLIII interaction in vivo, small molecules were assessed using a RepA-LacZ reporter gene assay. To conduct the assay, the gene encoding β-galactosidase was fused to the RepA gene, incorporated into commercially available plasmid pMU1550. E. coli JP3923 harboring plasmid pMU1550 was grown in minimal media (MM)/Tm(40 mg/mL) in the presence of various concentrations of the small molecules under evaluation. After reaching the appropriate optical density (˜OD600=0.6), Miller units of b-galactosidase were quantitated as per the standard protocol (see Miller, J. H, Experiments in Molecular Genetics, 1972, p352-355, Cold Spring Harbor Laboratory, Cold Spring Harbor, N.Y.). Any perturbation causing lower RepA production leads to a lowering of the β-galactosidase units upon quantitation A positive control utilizing a plasmid (pMU662) that overexpresses RNAII (containing the SLI-SLIII stretch) gave the expected high b-galactosidase signal. Each experiment was performed on at least three separate occasions. As shown in FIG. 5, evaluation of a number of small molecules showed apramycin, paromomycin I and kanamycin B to be potent compounds. Each of these compounds gave a dose dependent decrease in the reporter gene signal.

In a second control, erythromycin A, a general translation inhibitor, was tested using the reporter gene assay. Erythromycin A had no effect on the reporter gene assay, further indicating that apramycin and the other small molecules acted by mimicking plasmid incompatibility and not via some other mechanism.

Finally, additional controls were run in which the LacZ signal is upregulated (when the SLI-SLIII stretch is expressed on a second plasmid) and with no plasmid present. These experiments gave the expected results.

### EXAMPLE 2

### Assessment of Plasmid Elimination

The anti-plasmid effect of apramycin, paromomycin I and kanamycin B was assessed. In general, plasmid-containing E. coli resistant to ampicillin were cultured using growth media containing subinhibitory (sublethal) amounts of each of these compounds. The bacteria were allowed to reproduce for a defined number of generations, after which the bacteria were plated out onto agar plates containing the tested small molecules. Replicas were then made onto plates containing the tested small molecules and the antibiotic, ampicillin. Colonies that appeared on the master plate, but not the replicas had been rendered sensitive to ampicillin

In particular, E. coli strain JP4821 containing the commercially available IncB plasmid pMU2403, which encodes for β-lactamase, was grown in MM in the presence of 25 μg/mL apramycin for a defined number of generations. Typically, samples of the bacteria were plated out onto LB/Apra²⁵ plates after 10-12 generations, and replica plates were then made onto LB/Apra²⁵/Amp¹⁰⁰ plates. Percentage of plasmid lost was calculated by counting the number of colonies successfully replicated and dividing by the total number of colonies on the master plate; the entire plasmid loss assay was performed on two separate occasions. On average, greater than 200 master plate colonies were used for each of these evaluations. Evaluations of paromomycin I and kanamycin B were performed in an analogous fashion, with paromomycin I at 15 mg/mL, and kanamycin B at 10 mg/mL.

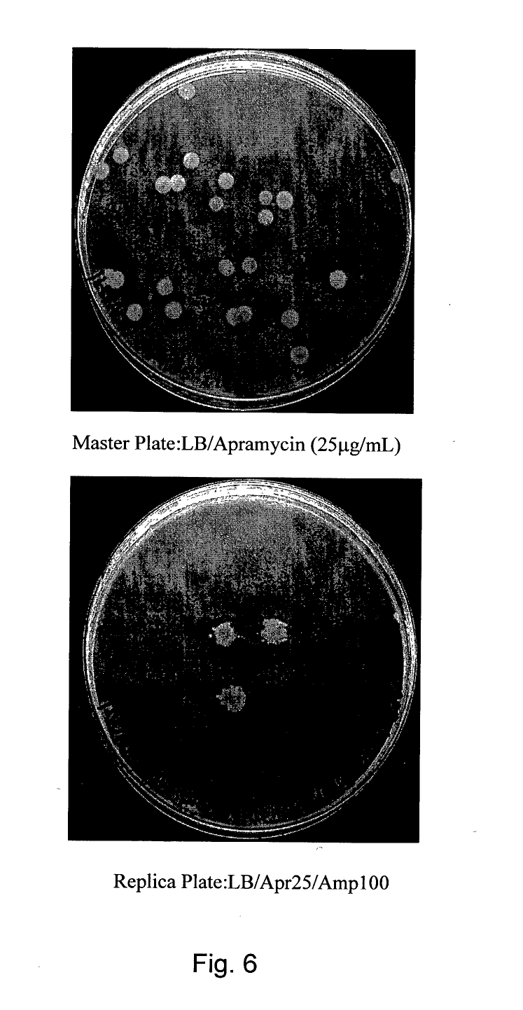

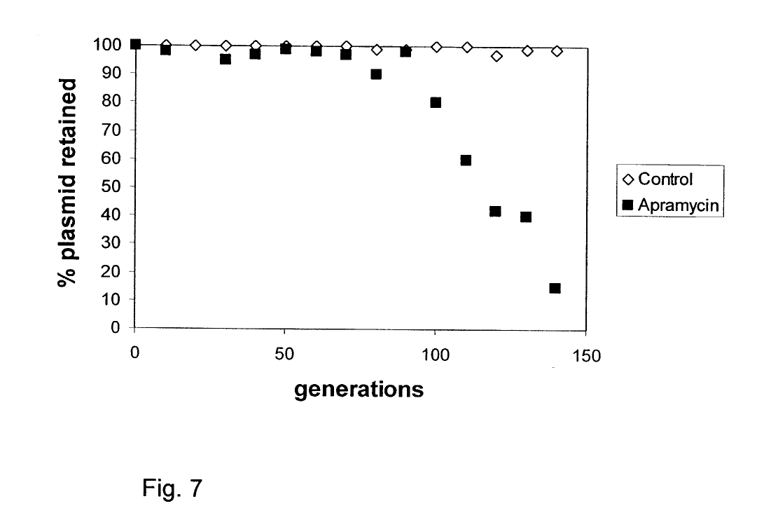

Master and replica plates after growth for 140 generations in the presence of apramycin are shown in FIG. 6. Results through 140 generations are indicated by the graph in FIG. 7. Apramycin caused virtually complete elimination after 220 generations.

To confirm that antibiotic sensitivity was due to plasmid loss, plasmid preparations were performed to isolate plasmid from the non-replicating colonies on the master plate, and from control colonies. No plasmids were isolated from the non-replicating colonies, while plasmid was successfully isolated from the control colonies that did replicate.

In addition, plasmid preparations performed on a large number of colonies that appeared on the apramycin/ampicillin plates showed that all of these colonies still contained the proper plasmid, indicating that resistance had not been transferred to the chromosome. Thus, apramycin causes virtually complete elimination of plasmid and sensitizes the bacteria to ampicillin, an antibiotic to which it was previously resistant. Kanamycin B and paromomycin I gave from 15-30% plasmid loss.

### EXAMPLE 3

### Assessment of Apramycin Interaction

To further investigate the mechanism by which apramycin causes plasmid elimination, the SLI-SLIII loop-loop interaction was reconstituted in vitro. SLI was biotinylated and bound to a streptavidin coated agarose bead. Fluorescein-labeled SLIII was added to form the loop-loop complex. This complex was then incubated with varous concentrations of apramycin and the amount of SLIII displaced by apramycin was assessed by quantitation of the fluorescence in solution. These experiments indicate that apramycin is disrupting the SLI-SLIII interaction with a Kd of 1 μM. This data is consistent with apramycin exerting its anti-plasmid effect by disruption of the SLI-SLIII interaction.

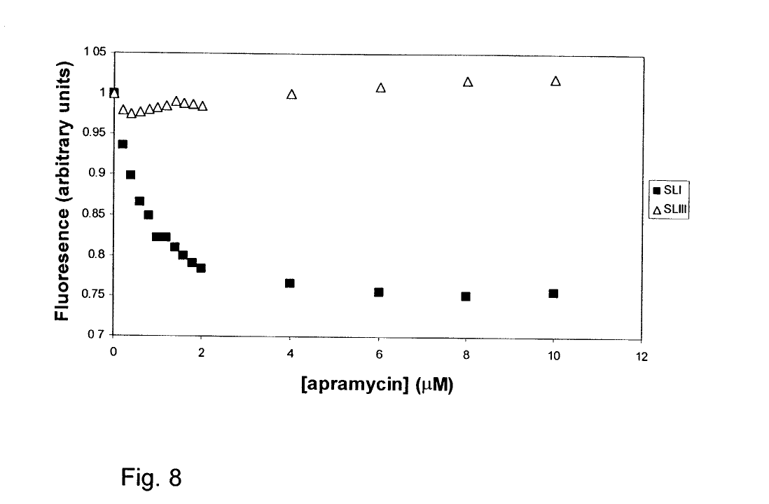

The direct binding of apramycin to SLI and SLIII was then assessed in vitro using fluorescently-labeled RNAs. When various apramycin concentrations were incubated with fluorescein-labeled SLIII, no significant change in fluorescence was observed (FIG. 8). However, incubation of fluorescently-labeled SLI with apramycin gave a significant decrease in the fluorescent signal (FIG. 8), and a Kd of 200 nM was calculated for the apramycin-SLI interaction. This data is consistent with apramycin exerting its anti-plasmid effect by binding to SLI.

For these experiments, RNA oligonucleotides were obtained from Dharmacon Research (Lafayette, Colo.), and were deprotected according to the manufacturer's protocol. The sequence of FI-SLI is FI.C.G.CCA.UAA.GCG.ACA.GCU.UGU.GGC. The sequence of FI-SLIII is FI.UAU.UUU.UCC.UCG.AAC.UUG.GCG.GAA.CGC.AGA.AAA.AUA. All fluorescence measurements were performed on an ISS PC1 spectrofluorometer with slitwidths and lamp current optimized for sufficient signal counts at the given RNA-fluorescein concentration. Deprotected RNA oligonucleotides were made up to 1 mM in 50 mM Tris-HCl, pH 7.3, and folded by heating to 90° C. for 3 min, then slowly cooling to rt. To assess the binding of apramycin to the fluorescently-labeled RNAs, 1500 mL of the folded RNA was transferred to a 4×10×48 mm fluorescence cuvette (Starna Cells) and allowed to equilibrate to 37° C. in the spectrofluorometer's sample chamber for 15 minutes. Apramycin was added in 5 mL portions from a stock solution, and a minimum of 5 minutes was allotted for equilibration. Three fluorescence spectra were acquired at each apramycin concentration. The spectra were taken with a 490 nm excitation wavelength and emission intensity was measured from 510-540 nm at an interval of one nanometer. At each point along the spectrum, an average of 3 data points was taken to give an averaged intensity at that integral wavelength. In processing the spectra, the point at 523 nm was chosen as a representative wavelength to monitor change in intensity over a range of apramycin concentrations. The 523 nm data points from each of the three averaged spectra were averaged at each apramycin concentration to give a composite average of nine measurements at 523 nm at each apramycin concentration. All intensity measurements are reported uncorrected for dilutions, as volume change over the course of the experiment is minimal.

These experimental results demonstrate that apramycin causes the loss of an IncB plasmid from E. coli. RNA binding experiments, and results from RNA-RNA loop-loop disruption assays, indicate that apramycin binds to SLI and disrupts the SLI-SLIII interaction. Reporter gene assays indicate that apramycin lowers the amount of RepA protein that is produced by the bacterial cell. Combined analysis of these experiments indicates that apramycin mimics the natural incompatibility determinant RNA I by binding to SLI, disrupting the SLI-SLIII loop-loop interaction, and thus preventing the translation of the RepA protein, inhibiting plasmid replication and ultimately leading to plasmid loss.

The treatment of drug-resistant microbes provided by the invention may be carried out by administering the antiplasmid composition in conjunction with any drug which, prior to sensitization of the microbe by the antiplasmid, had been rendered ineffective due to a plasmid-encoded resistance factor. Hence, any antibiotic composition may be used under these conditions.

In this aspect, therefore, the drug is selected from the group of antibiotics including beta-lactams, aminoglycosides, tetracyclines, sulfa drugs including sulfonamides and trimethoprim, lincosamides, glycopeptides, quinolones, aminocyclitols, lipopeptides, polypeptide antibiotics, nitroimidazoles, rifampicins, nitrofurans, oxazolidinones, trimethoprim, cloramphenicol, isoniazid, methenamine and mupirocin. These groups of antibiotics are well known in the art. See, e.g., the detailed description of classes and individual antibiotics contained in U.S. Pat. No. 6,406,880 (Thornton), incorporated herein. The classes of antibiotics and individual antibiotics, as well as antibiotic compounds with chemically homologous structures to the listed antibiotic compounds, are useful in the methods of the present invention when resistance to the antibiotic is encoded by a bacterial plasmid.

Antibiotics known to have significant activity against various microbes include, but are not limited to, e.g., amikacin, amoxicillin, azithromycin, capreomycin, cefinetazole, cefoxitin, ciprofloxacin, clarithromycin, clofazamine, cycloserine, dapsone, erythromycin, ethambutol (EMB), ethionamide, imipenem, isoniazid (INH), kanamycin, methicillin, minocycline, ofloxacin, para-amino salicylic acid, penicillin G, prothionamide, pyrazinamide (PZA), rifampin (RMP), rifabutin, sparfloxacin, sulfamethoxazole with trimethoprim, streptomycin (SM), tetracycline, thiacetazole, vancomycin and viomycin (C. B. INDERLIED ET AL., MANUAL OF CLINICAL MICROBIOLOGY 1385-1404 (P. R. Murray et al. eds., ASM Press 1995); A. KUCERS ET AL., THE USE OF ANTIBIOTICS 1352-1437 (J. B. Lippincott Co. 4th ed. 1987)).

By “beta-lactam” is meant any of the penicillin, cephalosporin, monobactam and carbapenem antibiotics having as a component of its structure the beta-lactam ring as understood in the art. (J. D. C YAO ET AL., MANUAL OF CLINICAL MICROBIOLOGY 1281-86 (Murray, P. R. et al. eds., ASM Press 1995); KUCERS, A. ET AL., THE USE OF ANTIBIOTICS 3-584 (J. B. Lippincott Co. 4th ed. 1987)).

By “penicillin” is meant an antibiotic having the 6-aminopenicillanic acid chemical nucleus as understood in the art (J. D. C YAO ET AL., MANUAL OF CLINICAL MICROBIOLOGY 1281-82 (Murray, P. R. et al. eds., ASM Press 1995)). Examples of penicillins include, but are not limited to, penicillin G, amoxicillin, methicillin, nafcillin, cloxacillin, dicloxacillin, oxacillin, ampicillin, bacampicillin, carbenicillin, ticarcillin, mezlocillin, and piperacillin, and especially aziocillin.

By “cephalosporin” is meant an antibiotic having the 7-aminocephalosporanic acid chemical nucleus as understood in the art (J. D. C YAO ET AL., MANUAL OF CLINICAL MICROBIOLOGY 1282-85 (Murray, P. R. et al. eds., ASM Press 1995)). Examples of cephalosporins useful in the methods of the invention include, but are not be limited to, cefadroxil, cefazolin, cephalexin, cephaloridine, cephalothin, cephamycin, cephapirin, cephradine, cefaclor, cefamandole, cefonicid, ceforanide, cefprozil, cefuroxime, loracarbef, cefinetazole, cefotetan, cefixime, cefotaxime, cefpodoxime, and ceftizoxime, and especially cefoxitin, cefoperazone, and ceftazidime, and most especially cettriaxone.

By “monobactam” is meant an antibiotic having the beta-lactam ring as the chemical nucleus, and having various side chains as understood in the art (J. D. C YAO ET AL., MANUAL OF CLINICAL MICROBIOLOGY 1285 (Murray, P. R. et al. eds., ASM Press 1995)). An example of a monobactam that is useful in the methods of the invention includes but is not limited to, aztreonam. It is reasonably expected that monobactams with chemical structures homologous to the above named monobactam compound will also be useful in the methods of the invention. By “carbapenem” is meant an antibiotic having the beta-lactam ring as the chemical nucleus, and having a hydroxyethyl side chain at the 6 position (in the trans configuration) and lacking a sulfur or oxygen atom in the nucleus as understood in the art (J. D. C YAO ET AL., MANUAL OF CLINICAL MICROBIOLOGY 1285-86 (Murray, P. R. et al. eds., ASM Press 1995)). Examples of carbapenems that are useful in the methods of the invention include, but are not limited to, imipenem, meropenem, panipenem, and biapenem.

By “beta-lactamase inhibitor” is meant an antibiotic having a modified beta-lactam structure as the chemical nucleus as understood in the art (J. D. C YAO ET AL., MANUAL OF CLINICAL MICROBIOLOGY 1286-87 (Murray, P. R. et al. eds., ASM Press 1995)). These compounds, having limited antibacterial activity in isolation, are known to act synergistically with the beta-lactams. Beta-lactamase inhibitors interfere with the enzymes that degrade beta-lactams (i.e., beta-lactamases). Microoorganisms can effectively evade the action of beta-lactam by using beta-lactamases, thus conferring resistance on the infectious agent. Thus, beta-lactamase inhibitors are useful in conjunction with the beta-lactam antibiotics, as adjuvants to beta-lactam therapy. Example of beta-lactamase inhibitors that are useful in the methods of the invention when a beta-lactam is also used, but are not limited to, clavulanic acid, sulbactam, and tazobactam.

By “aminoglycoside” or “aminocyclitol” is meant an antibiotic having amino sugars linked by glycosidic bonds to an aminocyclitol nucleus as understood in the art (J. D. C YAO ET AL., MANUAL OF CLINICAL MICROBIOLOGY 1287-88 (Murray, P. R. et al. eds., ASM Press 1995); KUCERS, A. ET AL., THE USE OF ANTIBIOTICS 585-750 (J. B. Lippincott Co. 4th ed. 1987)). Examples of aminoglycosides and aminocyclitols that are useful in the methods of the invention include, but are not limited to, streptomycin, kanamycin, gentamicin, tobramycin, amikacin, sisomicin, netilmicin, neomycin, framycetin and paromomycin.

By “quinolone” or “fluoroquinolone” is meant an antibiotic having a naphthyridine nucleus with different side chains as understood in the art (J. D. C YAO ET AL., MANUAL OF CLINICAL MICROBIOLOGY 1288-90 (Murray, P. R. et al. eds., ASM Press 1995); KUCERS, A. ET AL., THE USE OF ANTIBIOTICS 1203-75 (J. B. Lippincott Co. 4th ed. 1987)). Examples of quinolones that are useful in the methods of the invention include, but are not limited to, oxolinic acid, cinoxacin, flumequine, miloxacin, rosoxacin, pipemidic acid, norfloxacin, enoxacin, ciprofloxacin, ofloxacin, lomefloxacin, temafloxacin, fleroxacin, pefloxacin, amifloxacin, sparfloxacin, levofloxacin, clinafloxacin and especially nalidixic acid.

By “tetracycline” is meant an antibiotic having as a nucleus a hydronaphthacene structure as understood in the art (J. D. C YAO ET AL., MANUAL OF CLINICAL MICROBIOLOGY 1290-91 (Murray, P. R. et al. eds., ASM Press 1995); KUCERS, A. ET AL., THE USE OF ANTIBIOTICS 979-1044 (J. B. Lippincott Co. 4th ed. 1987)). Examples of tetracyclines that are useful in the methods of the invention include, but are not limited to, tetracycline, chlortetracycline, oxytetracycline, dimethylchlortetracycline demeclocycline, methacycline, lymecycline, clomocycline, doxycycline, and minocycline.

By “macrolide” is meant an antibiotic having a macrocyclic lactone ring with two attached sugars, desosamine and cladinose, and various substitutions as understood in the art (J. D. C YAO ET AL., MANUAL OF CLINICAL MICROBIOLOGY 1291-92 (Murray, P. R. et al. eds., ASM Press 1995); KUCERS, A. ET AL., THE USE OF ANTIBIOTICS 851-92 (J. B. Lippincott Co. 4th ed. 1987)). Examples of macrolides that are useful in the methods of the invention include, but are not limited to, erthromycin, oleandomycin, spiramycin, josamycin, rosaramicin, clarithromycin, azithromycin (also known as a azalide), dirithromycin, roxithromycin, flurithromycin, and rokitamycin.

By “lincosamide” is meant an antibiotic having an amino acid linked to an amino sugar as understood in the art (J. D. C YAO ET AL., MANUAL OF CLINICAL MICROBIOLOGY 1292-93 (Murray, P. R. et al. eds., ASM Press 1995); KUCERS, A. ET AL., THE USE OF ANTIBIOTICS 819-50 (J. B. Lippincott Co. 4th ed. 1987)). Examples of lincosamides that are useful in the methods of the invention include, but are not limited to, lincomycin and clindamycin.

By “glycopeptide” or “lipopeptide” is meant an antibiotic having a combination of peptide with either carbohydrate or lipid constituents, or both, as understood in the art (J. D. C YAO ET AL., MANUAL OF CLINICAL MICROBIOLOGY 1293 (Murray, P. R. et al. eds., ASM Press 1995); KUCERS, A. ET AL., THE USE OF ANTIBIOTICS 1045-72 (J. B. Lippincott Co. 4th ed. 1987)). Examples of glycopeptides and lipopeptides that are useful in the methods of the invention include, but are not limited to, vancomycin, teicoplanin, daptomycin (also known as YL 146032) and ramoplanin (also known as MDL 62198).

By a “polypeptide antibiotic” is meant an antibiotic having a cyclic polypeptide structure, or peptide linked amino acids, as understood in the art (J. D. C YAO ET AL., MANUAL OF CLINICAL MICROBIOLOGY 1295-96 (Murray, P. R. et al. eds., ASM Press 1995); KUCERS, A. ET AL., THE USE OF ANTIBIOTICS 899-913 (J. B. Lippincott Co. 4th ed. 1987)). Examples of polypeptide antibiotics that are useful in the methods of the invention include, but are not limited to, polymixins A, B, C, D and E, and bacitracin and gramicidin.

By “sulfa drugs” is meant any of a class of synthetic chemical substances derived from sulfanilamide, or para-aminobenzenesulfonamide. By “sulfonamide” is meant an antibiotic having a core structure similar to para-aminobenzoic acid as understood in the art, and by “trimethoprim” is meant an antibiotic that is a pyrimidine analog as understood in the art (J. D. C YAO ET AL., MANUAL OF CLINICAL MICROBIOLOGY 1293-95 (Murray, P. R. et al. eds., ASM Press 1995); KUCERS, A. ET AL., THE USE OF ANTIBIOTICS 1075-1117 (J. B. Lippincott Co. 4th ed. 1987)). Examples of sulfonamides that are useful in the methods of the invention include, but are not limited to, sulfanilamide, sulfacetarnide, sulfapyridine, sulfathiazole, sulfadiazine, sulfamerazine, sulfadimidine, sulfasomidine, sulfasalazine, mafenide, sulfamethoxazole, sulfamethoxypyridazine, sulfadimethoxine, sulfasymazine, sulfadoxine, sulfametopyrazine, sulfaguanidine, succinylsulfathiazole, and phthalylsulfathiazole. Trimethoprim is useful in the methods of the invention alone or in combination with any of the sulfonamides.

By “nitroimidazole” antibiotic is meant an antibiotic having a nitroimidazole nucleus as understood in the art (J. D. C YAO ET AL., MANUAL OF CLINICAL MICROBIOLOGY 1297 (Murray, P. R. et al. eds., ASM Press 1995); KUCERS, A. ET AL., THE USE OF ANTIBIOTICS 1290-1343 (J. B. Lippincott Co. 4th ed. 1987)). Examples of nitroimidazoles that are useful in the methods of the invention include, but are not limited to, metronidazole, tinidazole, nimorazole, ornidazole, camidazole, and secnidazole.

By “chloramphenicol” antibiotic is meant an antibiotic having a nitrobezene ring as its structural core as understood in the art (J. D. C YAO ET AL., MANUAL OF CLINICAL MICROBIOLOGY 1296-97 (Murray, P. R. et al. eds., ASM Press 1995); KUCERS, A. ET AL., THE USE OF ANTIBIOTICS 757-807 (J. B. Lippincott Co. 4th ed. 1987)). Examples of chloramphenicols that are useful in the methods of the invention include, but are not limited to, chloramphenicol and thiamphenicol.

By “rifampicin” is meant an antibiotic having an ansa, or macrocyclic, structural core (ansamycin antibiotics) as understood in the art (J. D. C YAO ET AL., MANUAL OF CLINICAL MICROBIOLOGY 1298 (Murray, P. R. et al. eds., ASM Press 1995); KUCERS, A. ET AL., THE USE OF ANTIBIOTICS 914-70 (J. B. Lippincott Co. 4th ed. 1987)). Examples of rifampicins that are useful in the methods of the invention include, but are not limited to, rifampin, rifamycin SV rifamycin B (rifamide) and rifabutin.

By “nitrofuran” is meant an antibiotic having a heterocyclic ring with a nitro group as understood in the art (J. D. C YAO ET AL., MANUAL OF CLINICAL MICROBIOLOGY 1298-99 (Murray, P. R. et al. eds., ASM Press 1995); KUCERS, A. ET AL., THE USE OF ANTIBIOTICS 1276-89 (J. B. Lippincott Co. 4th ed. 1987)). Examples of nitrofurans that are useful in the methods of the invention include, but are not limited to, nifuratel, nitrofurazone, furazolidone and nitrofurantoin.

By “methenamine” is meant an antibiotic having a tertiary amine as understood in the art (J. D. C YAO ET AL., MANUAL OF CLINICAL MICROBIOLOGY 1299 (Murray, P. R. et al. eds., ASM Press 1995); KUCERS, A. ET AL., THE USE OF ANTIBIOTICS 1344-48 (J. B. Lippincott Co. 4th ed. 1987)). Examples of tertiary amines that are useful in the methods of the invention include, but are not limited to, methenamine, mandelate, methenamine hippurate.

By “mupirocin” (also known as pseudomonic acid) is meant an antibiotic having a unique 9-hydroxy-nonanoic acid moiety as understood in the art (Yao, J. D. C. et al., In: Murray, P. R. et al., eds. Manual of Clinical Microbiology, ASM Press, Washington, D.C. (1995) pp.1299-1300; and KUCERS, A. ET AL., THE USE OF ANTIBIOTICS 4th ed J. B. Lippincott Co. Philadelphia, Pa. (1987) pp.754-756). J. D. C YAO ET AL., MANUAL OF CLINICAL MICROBIOLOGY 1299-1300 (Murray, P. R. et al. eds., ASM Press 1995); KUCERS, A. ET AL., THE USE OF ANTIBIOTICS 754-56 (J. B. Lippincott Co. 4th ed. 1987)).

The antiplasmid compositions and antibiotics useful in the methods of the present invention may be formulated into pharmaceutical compositions or similar forms and administered by any means that will deliver a therapeutically effective dose. Hence, they may be included together in a combined pharmaceutical formulation or administered as part of a kit or regimen in which an effective amount of the antiplasmid composition is administered in a first dosage form and an effective amount of a drug or drugs to which the microbe is sensitized by the antiplasmid composition is administered in a second dosage form. Such compositions can be administered orally, parenterally, by inhalation spray, rectally, intradermally, transdermally, or topically in dosage unit formulations containing conventional nontoxic pharmaceutically acceptable excipients, carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer'solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the antiplasmid composition and antibiotic discussed herein can be prepared by mixing the active agent or agents with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of antiplasmid composition or antibiotic, in combination with one another or separately, that is combined with the carrier materials to produce a single dosage will vary depending upon the patient and the particular mode of administration. Amounts and regimens for the administration of a given antiplasmid composition and a given antibiotic can be determined readily by those with ordinary skill in the clinical art of treating such microbial infections. For example, the concentration of a given antibiotic will depend on the antibiotic used. The antibiotics may be provided in the methods of the invention at those doses known in the art to be therapeutic. Generally, the dosage of the antibiotic and of the antiplasmid composition will vary depending upon additional considerations relating to the condition of the subject such as: age; health; conditions being treated; kind of concurrent treatment, if any; frequency of treatment and the nature of the effect desired; extent of tissue damage; gender; duration of the symptoms; and, contraindications, if any, and other variables to be adjusted by the individual physician. Dosages can be administered in one or more applications to obtain the desired results.

Those skilled in the art will appreciate that antibiotic dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

All publications, patents and patent applications cited in the specification are herein incorporated by reference as if each individual publication or patent application was specifically and individually indicated to be incorporated by reference.

What is claimed is:

1. A method of treating a drug-resistant bacterial infection, including a multi-drug resistant infection in a subject suffering from said infection, said method comprising:
(a) administering to the subject an effective amount of an antiplasmid composition that mimics plasmid incompatibility, thereby rendering the drug-resistant bacteria sensitive to the drug(s) for which the resistance is plasmid-mediated;
(b) administering to the subject an effective amount of the drug(s) to which the bacteria had been sensitized by the method of (a).

2. The method of claim 1, wherein the drug-resistant bacterial infection is caused by MRSA or VRE.

3. The method of claim 1, wherein the drug is selected from the group consisting of beta-lactams, aminoglycosides, tetracyclins, macrolides, sulfa drugs, lincosamides, glycopeptides, quinolones, aminocyclitols, lipopeptides, polypeptide antibiotics, nitroimidazoles, rifampicins, nitrofurans, oxazolidinones, trimethoprim, cloramphenicol, isoniazid, methenamine and mupirocin.

4. The method of claim 1, wherein the subject is a mammal.

5. The method of claim 4, wherein the subject is a human.

6. The method of claim 1, wherein the composition is administered by subcutaneous injection, intramuscular injection, intravenously, inhalation spray, topically, or orally.

7. The method of claim 1, wherein the composition comprises an aminoglycoside.

8. The method of claim 7, wherein the composition is selected from the group consisting of apramycin, tobramycin, paromomycin I, kanamycin B, and derivatives thereof.

9. The method of claim 1 wherein the effective amount of the antiplasmid composition comprises a subinhibitory dose of the composition.

10. The method of claim 1 wherein the composition mimics plasmid incompatibility by inhibiting Rep protein activity.

11. The method of claim 1 wherein the composition mimics plasmid incompatibility by disrupting a stem-loop interaction between an RNA primer required for plasmid replication and its antisense transcript.

12. The method of claim 11 wherein the composition disrupts stem-loop interaction by binding to at least a portion of a plasmid YUNR consensus sequence.

13. A method of screening compositions for the ability to interfere with plasmid replication by mimicking plasmid incompatibility, said method comprising screening the compositions for the ability to inhibit Rep protein activity.

14. The method of claim 13, wherein the Rep protein activity comprises binding of the Rep protein to plasmid DNA sequences.

15. A method of screening compositions for the ability to interfere with plasmid replication by mimicking plasmid incompatibility, said method comprising screening the compositions for the ability to disrupt a stem-loop interaction between an RNA primer required for the plasmid replication and its antisense transcript.

16. A pharmaceutical composition for the treatment of drug-resistant microbes comprising an effective amount of an antiplasmid composition that mimics plasmid incompatibility, thereby rendering the the drug resistant microbe sensitive to a drug for which resistance is plasmid-mediated and an effective amount of a drug to which the microbe is sensitized by the antiplasmid composition.

17. A pharmaceutical composition as set forth in claim 16 comprising at least one antiplasmid composition identified by screening compositions for the ability to inhibit Rep protein activity.

18. A pharmaceutical composition as set forth in claim 16 comprising at least one antiplasmid composition identified by screening compositions for the ability to disrupt a stem-loop interaction between an RNA primer required for the plasmid replication and its antisense transcript.

19. A kit for the treatment of drug-resistant microbes comprising a first dosage form comprising an effective amount of an antiplasmid composition that mimics plasmid incompatibility, thereby rendering the drug resistant microbe sensitive to a drug for which resistance is plasmid-mediated and a second dosage form comprising an effective amount of a drug to which the microbe is sensitized by the antiplasmid composition.

20. A kit as set forth in claim 19 comprising at least one antiplasmid composition identified by screening compositions for the ability to inhibit Rep protein activity.

21. A kit as set forth in claim 19 comprising at least one antiplasmid composition identified by screening compositions for the ability to disrupt a stem-loop interaction between an RNA primer required for the plasmid replication and its antisense transcript.


SEQUENCE LISTING (2 sequences)

SEQ ID NO: 1 - RNA, 23 nt, Escherichia coli
  misc_feature (1)..(1): Fluorscein labelled
cgccauaagcgacagcuuguggc

SEQ ID NO: 2 - RNA, 36 nt, Escherichia coli
  misc_feature (1)..(1): Fluorescein labelled
uauuuuuccucgaacuuggcggaacgcagaaaaaua